(19) United States
(12) Patent Application Publication
(10) Pub. No.: US 2023/0372251 A1
(43) Pub. Date: Nov. 23, 2023

(54) FAST DISSOLVING THERMAL GEL HYPROMELLOSE HOLLOW CAPSULE AND METHOD FOR MAKING THE SAME

(71) Applicant: LEFAN CAPSULE INTERNATIONAL INC, Ontario, CA (US)

(72) Inventors: Shenghui CAO, Changzhou (CN); Peiyong LIU, Zhenjiang (CN); Yiling CHEN, Zhenjiang (CN); Hanyu CAO, Zhenjiang (CN); Menglin TAN, Zhenjiang (CN); Haolong MENG, Zhenjiang (CN); Zhongming FANG, Zhenjiang (CN)

(21) Appl. No.: 17/664,098

(22) Filed: May 19, 2022

(51) Int. Cl. A61K 9/48

(52) U.S. Cl. CPC A61K 9/4816; A61K 9/485

(57) ABSTRACT

A fast disintegrating thermally gelling hydroxypropyl methylcellulose (Hypromellose, HPMC) empty capsule can be formed from HPMC 2910 that has a methoxyl content of 27.0% to 30.0% (w/w) and a hydroxylpropoxyl content of 7.0% to 12.0% (w/w). Except for adding a small amount of opacifier and/or colorant, according to customer requirements, the composition can be free of any plasticizer, moisturizing agent, stabilizer, coagulant and other auxiliary materials. The resulting capsule dipped by thermal gelation method has no cracks or wrinkles. The resulting capsule exhibits rapid disintegration release, independent of any dissolution medium, that is significantly faster than capsules dipped by thermal gelation using Hypromellose 2906.

### BACKGROUND OF THE INVENTION

### 1. Field of the Invention

Embodiments of the invention relate generally to pharmaceutical excipients and encapsulation. More particularly, embodiments of the invention relate a fast dissolving thermal gel hydroxypropyl cellulose (Hypromellose or HPMC) hollow capsule and methods for making the same.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

HPMC is a polymeric compound that contain repeating units of hydroxypropyl methylcellulose. The properties of HPMC can vary greatly and are defined by their molecular weight, the percentage of hydroxyl groups, the percentage of hydroxypropyl groups, and viscosity measurements. HPMC is found a broad variety of commercial products such as food additives, excipients and lubricants.

In the pharmaceutical industry, Hypromellose polymers are used in solid oral dosage forms as a binder, a film coating, and a controlled-release matrix. Hypromellose polymers are used in the preparation of hydrophilic matrix tablets because of their rapid formation of a uniform, strong, and viscous gel layer, which protects the matrix from disintegration and controls the rate of drug release.

HPMC is available in several types with different degrees of substitution (DS) and molar substitution (MS). Further, the added hydroxypropyl group introduces a secondary hydroxyl group that can also be etherified during the preparation of Hypromellose polymers, giving rise to possible additional chain extension. Hypromellose is a partly O-methylated and O-(2-hydroxypropylated) cellulose ether derivative having formula (I), below:

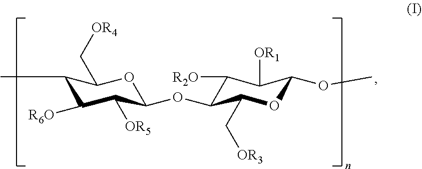

where R₁-R₆ are each independently —H, —CH₃ or —CH₂CH(OH)CH₃.

Currently, HPMC is defined by the different substitution types using a four-digit number. For example, Hypromellose 2208, Hypromellose 2906, and Hypromellose 2910, where the first two digits refer to the approximate percentage content of the methoxy group (—OCH₃), and the second two digits refer to the approximate percentage content of the hydroxypropoxy group (—OCH₂CH(OH)CH₃). Such variations in the ratios of methoxy and hydroxypropoxyl substitutions, as well as the polymer's molecular weight, affect their properties such as organic solubility, thermal gelation temperature in aqueous solution, swelling, diffusion, and drug-release rate. Typically, HPMC can be made in many different viscosity grades corresponding to average molecular weights from 80,000 to 1,200,000 Dalton (Da).

Attempts have been made to use Hypromellose for capsule formulation. The HPMC empty capsules prepared by a conventional thermal gelation method typically will include plasticizers, moisturizing agents, stabilizers and other auxiliary materials, and it is hard to truly achieve a pure HPMC capsule without adding any auxiliary materials. The empty capsules prepared conventionally by the thermal gelation method of Hypromellose 2906 will have a small amount of cracks, wrinkles and other defects. These empty capsules can further require a significant amount of time for the complete dissolution of the capsule shell as compared to conventional gelatin empty capsules.

In view of the foregoing, there is a need for improved empty capsules that can present with rapid dissolving times.

### SUMMARY OF THE INVENTION

Embodiments of the present invention provide a capsule composition comprising hydroxypropyl methylcellulose (HPMC) having 27.0% to 30.0% by weight methoxy content and 7.0% to 12.0% by weight hydroxypropoxy content; and water.

Embodiments of the present invention further provide a method for making a capsule comprising mixing hydroxypropyl methylcellulose (HPMC) with water while stirring a resulting mixture, the HPMC having 27.0% to 30.0% by weight methoxy content and 7.0% to 12.0% by weight hydroxypropoxy content; heating the resulting mixture to greater than 75° C. to create a dispersed HPMC aqueous dispersion; adding cooled water to the dispersed HPMC aqueous dispersion to cool the dispersed HPMC aqueous dispersion to form a cooled dispersed HPMC aqueous dispersion at a temperature from 10° C. to 15° C.; allowing the cooled dispersed HPMC aqueous dispersion to stand to form a dissolved HPMC aqueous solution; heating the dissolved HPMC aqueous solution to 30 to 50° C. to create a heated dissolved HPMC aqueous solution; immersing a capsule mold into the heated dissolved HPMC aqueous solution; permitting the mold to dry in a first drying step at a temperature of 40 to 80° C. and a relative humidity of greater than 60 percent; and permitting the mold to dry in a second drying step at a temperature of 25 to 50° C. and a relative humidity of 20 to 60 percent.

Embodiments of the present invention also provide an empty capsule comprising hydroxypropyl methylcellulose (HPMC) having 27.0% to 30.0% by weight methoxy content and 7.0% to 12.0% by weight hydroxypropoxy content; and water, wherein the capsule composition is free of any plasticizer, moisturizing agent, stabilizer, and coagulant.

These and other features, aspects and advantages of the present invention will become better understood with reference to the following drawings, description and claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any composition or production method, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a fast disintegrating thermally gelling hydroxypropyl methylcellulose (Hypromellose, HPMC) empty capsule and its preparation method. The empty capsule can be formed from HPMC 2910 that has a methoxyl content of 27.0% to 30.0% (w/w) and a hydroxylpropoxyl content of 7.0% to 12.0% (w/w). Except for adding a small amount of opacifier and/or colorant, according to customer requirements, the composition can be free of any plasticizer, moisturizing agent, stabilizer, coagulant and other auxiliary materials. The resulting capsule dipped by thermal gelation method has no cracks or wrinkles. The resulting capsule exhibits rapid disintegration release, independent of any dissolution medium, that is significantly faster than capsules dipped by thermal gelation using Hypromellose 2906.

In some embodiments, a composition for forming an empty capsule can include the following components:
(a) 200 parts of HPMC 2910, having 27.0% to 30.0% (w/w) methoxy content and 7.0% to 12.0% (w/w) hydroxypropoxy content. The HPMC can have an average molecular weight from 25000 to 80000 Da, typically from 30000 to 50000 Da;
(b) 650 to 1000 parts water;
(c) 0 to 6 parts of an opacifier which can be selected from, for example, titanium dioxide, calcium carbonate, calcium hydrogen phosphate, aluminum hydroxide, barium sulfate, talc, zinc oxide and magnesium oxide; and
(d) 0 to 2 parts of a colorant which can be selected from, for example, one or more of synthetic pigments, iron oxide and natural pigment. As used herein, “parts” refer to parts by weight.

To make a composition for forming an empty capsule, 200 parts of HPMC 2910 (27.0% to 30.0% (w/w) methoxyl content and 7.0% to 12.0% (w/w) hydroxypropoxyl content), is disperse at >75° C., typically 80 to 90° C., with 650 to 1000 parts of water, typically thermally purified water. The resulting mixture is stirred, slowly, for 60 to 100 minutes at a rotational speed of 20 to 30 rpm while maintaining the temperature greater than 75° C., typically between 80 and 90° C.

The resulting dispersed HPMC aqueous dispersion is cooled with cooling water, having a temperature of about 10° C., while stirring is continued, typically at 20 to 30 rpm, during the cooling process. The temperature of the HPMC aqueous dispersion is lowered to 10 to 25° C., typically 10 to 15° C., and allowed to stand for 10 to 15 hours to completely dissolve into a transparent HPMC aqueous solution.

The resulting HPMC aqueous solution is warmed to 30 to 50° C., typically 35 to 45° C., and slowly stirred at a speed of 20 to 30 rpm during the heating process. During the heating process of the HPMC aqueous solution, any desired colorant can be added. The temperature of the HPMC aqueous solution can be increased to 30 to 50° C., typically 35˜45° C., and the viscosity of the HPMC aqueous solution is adjusted, with water, to 500 to 10000 mPa·s, typically 1000 to 3500 mPa·s, and the solution can be placed in a gelatin basin for later use.

The surface temperature of a die pin on a capsule mold is preheated to 65 to 140° C., typically 80 to 120° C., and the preheated die pin is immersed in the gelatin basin containing the HPMC aqueous solution at 30 to 50° C., typically 35 to 45° C. The mold, soaked in the HPMC aqueous solution, is placed, for 30 to 60 minutes, into an environment having a temperature of 40 to 80° C., typically 50 to 60° C., and a relative humidity of greater than 60%, typically 60 to 70%. The mold is then transferred to an environment where the temperature is 25 to 50° C., typically 30 to 40° C., and the relative humidity is 20 to 60%, typically 35 to 45%, to deep dry for 30 to 60 minutes in order to obtain capsules with suitable specifications and sizes without crack and/or wrinkle defects of the capsules.

The dissolution situation of a size 0 HPMC empty capsule prepared by the above process was evaluated. Using the slurry method, with a stirring speed of 100rpm, under the conditions of 500 ml medium and temperature of 37.0±1° C., the dissolution of size 0 HPMC empty capsule prepared according to embodiments of the present invention is compared with the dissolution of conventional HPMC empty capsules prepared by HPMC 2906 thermal gelation method according to conventional methods as described in U.S. Pat. No. 9,655,860, the contents of which are herein incorporated by reference.

The results are shown in the following table:

| | HPMC 2910 empty capsulations | | Conventional | |
|---|---|---|---|---|
| | formed from the formulation of | | HPMC 2906 | |
| | the present invention | | Empty Capsules | |
| | | Completely | | Completely |
| | Start to crack | dissolved | Start to crack | dissolved |
| Water | 4'21″ | 14'51″ | 5'36″ | 19'51″ |
| medium | | | | |
| PH 6.0 | 4'00″ | 15'49″ | 5'08″ | 20'25″ |
| Potassium | | | | |
| dihydrogen | | | | |
| phosphate | | | | |
| buffer | | | | |
| solution | | | | |
| PH 4.5 | 3'57″ | 13'57″ | 5'52″ | 21'10″ |
| Potassium | | | | |
| dihydrogen | | | | |
| phosphate | | | | |
| buffer | | | | |
| solution | | | | |
| PH 1.2 | 4'01″ | 14'23″ | 5'28″ | 19'36″ |
| Hydrochloric | | | | |
| acid solution | | | | |

As can be seen, in water, the HPMC 2910 according to embodiments of the present invention started to crack at 4 minutes, 21 seconds, while the conventional capsule took about 1 minute, 15 seconds longer. The capsule according to the present invention completely dissolved in 14 minutes, 51 seconds, while the conventional capsule according to the comparison example took 5 minutes longer to completely dissolve. Similar results were observed in more acidic conditions.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A capsule composition comprising:
hydroxypropyl methylcellulose (HPMC) having 27.0% to 30.0% by weight methoxy content and 7.0% to 12.0% by weight hydroxypropoxy content; and
water.

2. The capsule composition of claim 1, wherein water is present at from about 3.5 to about 5 times the amount, by weight, of HPMC.

3. The capsule composition of claim 1, wherein the capsule composition is free of any plasticizer, moisturizing agent, stabilizer, and coagulant.

4. The capsule composition of claim 1, wherein the capsule composition has a viscosity from 500 to 10000 mPa·s at 30 to 50° C.

5. The capsule composition of claim 1, further comprising an opacifier.

6. The capsule composition of claim 5, wherein the opacifier is selected from the group consisting of titanium dioxide, calcium carbonate, calcium hydrogen phosphate, aluminum hydroxide, barium sulfate, talc, zinc oxide and magnesium oxide.

7. The capsule composition of claim 5, wherein the opacifier is present in from 0.5 to 0.7 percent by weight.

8. The capsule composition of claim 1, further comprising a colorant.

9. The capsule composition of claim 8, wherein the colorant is selected from the group consisting of synthetic pigments, iron oxide and natural pigment.

10. A method for making a capsule comprising:
mixing hydroxypropyl methylcellulose (HPMC) with water while stirring a resulting mixture, the HPMC having 27.0% to 30.0% by weight methoxy content and 7.0% to 12.0% by weight hydroxypropoxy content;
heating the resulting mixture to greater than 75° C. to create a dispersed HPMC aqueous dispersion;
adding cooled water to the dispersed HPMC aqueous dispersion to cool the dispersed HPMC aqueous dispersion to form a cooled dispersed HPMC aqueous dispersion at a temperature from 10° C. to 15° C.;
allowing the cooled dispersed HPMC aqueous dispersion to stand to form a dissolved HPMC aqueous solution;
heating the dissolved HPMC aqueous solution to 30 to 50° C. to create a heated dissolved HPMC aqueous solution;
immersing a capsule mold into the heated dissolved HPMC aqueous solution;
permitting the mold to dry in a first drying step at a temperature of 40 to 80° C. and a relative humidity of greater than 60 percent; and
permitting the mold to dry in a second drying step at a temperature of 25 to 50° C. and a relative humidity of 20 to 60 percent.

11. The method of claim 10, wherein the resulting mixture is stirred for 60 to 100 minutes at a rotational speed of 20 to 30 rpm.

12. The method of claim 10, wherein the cooling water has a temperature of about 10° C.

13. The method of claim 10, wherein the cooled dispersed HPMC aqueous dispersion stands for 10 to 15 hours to form the dissolved HPMC aqueous solution.

14. The method of claim 10, further comprising adding a colorant to the heated dissolved HPMC aqueous solution.

15. The method of claim 10, further comprising adjusting the viscosity of the heated dissolved HPMC aqueous solution to 500˜10000mpa˜s at 30 to 50° C.

16. The method of claim 10, further comprising moving the heated dissolved HPMC aqueous solution to a gelatin basin.

17. The method of claim 16, further comprising warming the heated dissolved HPMC aqueous solution in the gelatin basin to 30 to 50° prior to immersing the capsule mold therein.

18. The method of claim 10, further comprising heating a surface temperature of a die pin on the capsule mold to 65 to 140° C. prior to immersing the capsule mold into the heated dissolved HPMC aqueous solution.

19. An empty capsule comprising:
hydroxypropyl methylcellulose (HPMC) having 27.0% to 30.0% by weight methoxy content and 7.0% to 12.0% by weight hydroxypropoxy content; and
water,
wherein the capsule composition is free of any plasticizer, moisturizing agent, stabilizer, and coagulant.
